(19) United States
(12) Patent Application Publication
(10) Pub. No.: US 2015/0253307 A1
(43) Pub. Date: Sep. 10, 2015

(54) POLYMERIC FIBER-SCAFFOLDED ENGINEERED TISSUES AND USES THEREOF

(71) Applicants: Kevin PARKER, Waltham, MA (US); PRESIDENT AND FELLOWS OF HARVARD COLLEGE, Cambridge, MA (US)

(72) Inventors: Kevin Kit Parker, Cambridge, MA (US); Mohammad Reza Badrossamay, Irvine, CA (US); Ashutosh Agarwal, Miami Beach, FL (US)

(73) Assignee: PRESIDENT AND FELLOWS OF HARVARD COLLEGE, Cambridge, MA (US)

(21) Appl. No.: 14/429,826

(22) PCT Filed: Sep. 20, 2013
(86) PCT No.: PCT/US2013/060823

§ 371 Date: Mar. 20, 2015

Related U.S. Application Data
(60) Provisional application No. 61/704,049, filed on Sep. 21, 2012.

(51) Int. Cl. G01N 33/50; C12N 5/071

(52) U.S. Cl. CPC G01N 33/5082; C12N 5/0697; C12N 2533/30; C12N 2513/00

(57) ABSTRACT

The present invention provides devices, constructs, and methods of use of polymeric fiber-scaffolded engineered tissues and assays for identifying compounds that modulate a contractile function, using such devices and constructs.

### RELATED APPLICATIONS

This application claims priority to U.S. Provisional Patent Application Ser. No. 61/704,049, filed on Sep. 21, 2012, the entire contents of which are incorporated herein by reference.

### BACKGROUND OF THE INVENTION

Identification and evaluation of new therapeutic agents or identification of suspect disease associated targets typically employ animal models which are expensive, time consuming, require skilled animal-trained staff and utilize large numbers of animals. In vitro alternatives have relied on the use of conventional cell culture systems which are limited in that they do not allow the three-dimensional interactions that occur between cells and their surrounding tissue. This is a serious disadvantage as such interactions are well documented as having a significant influence on the growth and activity of cells in vivo since in vivo cells divide and interconnect in the formation of complex biological systems creating structure-function hierarchies that range from the nanometer to meter scales.

Efforts to build biosynthetic materials or engineered tissues that recapitulate these structure-function relationships often fail because of the inability to replicate the in vivo conditions that coax this behavior from ensembles of cells. For example, engineering a functional muscle tissue requires that the sarcomere and myofibrillogenesis be controlled at the micron length scale, while cellular alignment and formation of the continuous tissue require organizational cues over the millimeter to centimeter length scale. Thus, to build a functional biosynthetic material, the biotic-abiotic interface must contain the chemical and mechanical properties that support multi-scale coupling.

Accordingly, there is a need for improved methods and systems that replicate the in vivo environment and that may be used for, for example, determining the effect of a test compound on biological relevant parameters in order to enhance and speed-up the drug discovery and development process.

### SUMMARY OF THE INVENTION

Described herein are devices, constructs and methods for measurements of physiologically relevant properties of in vitro tissue constructs. The devices of the present invention can be used in, for example, screening assays, e.g., high-throughput screening assays, to determine the effects of a test compound on living tissue by examining the effect of the test compound on various biological responses, such as for example, cell viability, cell growth, migration, differentiation and maintenance of cell phenotype, electrophysiology, metabolic activity, muscle cell contraction, osmotic swelling, structural remodeling and tissue level pre-stress.

Accordingly, in one aspect, the present invention provides devices for measuring a contractile function. The devices include a solid support structure, and a strip of co-cultured muscle tissue adhered to the solid support structure, wherein the co-cultured muscle tissue comprises a layer of isolated cells seeded on a sheet of aligned polymeric fibers comprising a biogenic polymer, and a hydrogel layer comprising cells coated on the polymeric fiber layer, wherein the strip of co-cultured muscle tissue can perform a contractile function.

In another aspect, the present invention provides constructs for producing a polymeric fiber-scaffolded engineered tissue. The constructs include a support structure, a sheet of aligned polymeric fibers on the support structure, wherein the aligned polymeric fibers comprise a biogenic polymer, cells seeded on the aligned polymeric fiber layer, and a hydrogel comprising cells coated on the aligned polymeric fiber layer seeded with cells.

In one aspect, the present invention provides methods for fabricating a polymeric fiber-scaffolded engineered tissue. The methods include providing a solid support structure, providing a sheet of aligned polymeric fibers on the solid support structure, wherein the aligned polymeric fibers comprise an extracellular matrix protein, seeding cells on the aligned polymeric fiber layer, applying a hydrogel comprising cells on the cells seeded on the sheet of aligned polymeric fibers, culturing the cells to form a tissue; and removing a portion of said formed tissue thereby generating strips of said formed tissue adhered at one end to said solid support structure.

The present invention also provides polymeric fiber-scaffolded engineered tissues prepared according to the methods of the invention.

In one embodiment, the devices comprise a plurality of strips of the co-cultured muscle tissue.

In one embodiment, the methods include producing a plurality of strips of the co-cultured muscle tissue.

The cells on the aligned polymeric fiber sheet and in the hydrogel may be of the same type or different types.

In one embodiment, the cells are myocytes, such as cardiomyocytes. In another embodiment, the cells are smooth muscle cells or striated muscle cells. In yet another embodiment, the cells are muscle satellite cells. In one embodiment, the cells on the aligned polymeric fiber sheet are skeletal muscle cells and the cells in the hydrogel are muscle satellite cells.

The solid support structure may be a glass coverslip, a Petri dish, a strip of glass, a glass slide, or a multi-well plate. The solid support structure may comprise one or more microfluidics chambers. In one embodiment, the one or more microfluidics chambers are operable connected to one or more inlet microchannels and one or more outlet microchannels.

In one embodiment, the solid support structure further comprises an optical signal capture device; and an image processing software to calculate change in an optical signal. In one embodiment, the optical signal capture device comprises fiber optic cables in contact with said culture wells.

In one embodiment, the aligned polymeric fiber sheet is prepared by rotary jet-spinning of an extracellular matrix protein.

In one embodiment, the biogenic polymer is a protein, a polysaccharide, a lipid, a nucleic acid, or a combination thereof. The protein may be a fibrous protein, such as an extracellular matrix protein. In one embodiment, the extracellular matrix protein is selected from the group consisting of silk, a keratin, an elastin, a fibrillin, a fibrinogen, a fibrin, a thrombin, a fibronectin, a laminin, a collagen, a vimentin, a neurofilament, an amyloid, an actin, a myosin, and a titin. In one embodiment, the polymeric fiber is a biohybrid fiber.

The hydrogel may comprise a substance selected from the group consisting of gelatin, collagen, arginine, fibrin, fibronectin, glucose, and glycoprotein, or a combination thereof.

In one aspect, the present invention provides methods for identifying a compound that modulates a contractile function. The methods include providing a polymeric fiber-scaffolded engineered tissue, contacting the polymeric fiber-scaffolded engineered tissue with a test compound; and determining the effect of the test compound on a contractile function in the presence and absence of the test compound, wherein a modulation of the contractile function in the presence of said test compound as compared to the contractile function in the absence of said test compound indicates that said test compound modulates a contractile function, thereby identifying a compound that modulates a contractile function.

In another aspect, the present invention provides methods for identifying a compound useful for treating or preventing a muscle disease. The methods include providing a polymeric fiber-scaffolded engineered tissue, contacting the polymeric fiber-scaffolded engineered tissue with a test compound, and determining the effect of the test compound on a contractile function in the presence and absence of the test compound, wherein a modulation of the contractile function in the presence of said test compound as compared to the contractile function in the absence of said test compound indicates that said test compound modulates a contractile function, thereby identifying a compound useful for treating or preventing a muscle disease.

The contractile function may be a biomechanical activity, such as contractility, cell stress, cell swelling, and rigidity. In one embodiment, the contractile function is an electrophysiological activity. In one embodiment, the electrophysiological activity is a voltage parameter selected from the group consisting of action potential, action potential duration (APD), conduction velocity (CV), refractory period, wavelength, restitution, bradycardia, tachycardia, and reentrant arrhythmia. In another embodiment, the electrophysiological activity is a calcium flux parameter selected from the group consisting of intracellular calcium transient, transient amplitude, rise time (contraction), decay time (relaxation), total area under the transient (force), restitution, focal and spontaneous calcium release.

In one embodiment, the methods further comprise applying a stimulus to the polymeric fiber-scaffolded engineered tissue.

### BRIEF DESCRIPTION OF THE DRAWINGS

FIGS. 1A-1H depict high throughput contractility experiments using the Muscle This Film (MTF) platform described in U.S. Patent Publication Nos. 2009/0317852 and 2012/0142556, the entire contents of each of which are incorporated herein by reference. (A) Immunostain image of mouse embryonic stem cell derived cardiomyocytes patterned in 20 μm lines with 20 μm spacing (scale bar=10 μm), medium gray—DAPI, dark gray—sarcomeres, light gray—actin. (B) Brightfield image of a muscular thin film (MTF) chip comprising 39 individual engineered neonatal rat ventricular myocytes MTFs (scale bar=1 mm) used in contractility assays. (C) Brightfield image of an MTF chip comprising 8 individual engineered mES tissue MTFs (scale bar=2 mm), medium gray—film length outline, dark gray—peak systolic film length. (D) Stress traces for the chip in panel (C) paced at 3 Hz. (E) Peak systolic, diastolic, and twitch stress for five cell types (n=6-15 for all cell types). Validation of the fluidic heart on a chip technology by comparing isoproterenol dose response on contraction for (F) ex vivo rat ventricular myocardium strips (N=4); MEAN±SEM, *P<0.05, **P<0.01 vs. baseline, (G) in vitro neonatal cardiac MTFs in an open bath configuration (N=25 MTFs from the same chip); MEAN±SEM, and (H) in vitro neonatal cardiac MTFs in an enclosed fluidic device (N=19 MTFs from the same chip); MEAN±SEM.

FIGS. 2A-2D depict an exemplary device for the fabrication of aligned polymeric fiber sheets or scaffolds for cell culture and the results of cell culture experiments using the same. (A) An exemplary device employing rotational motion for the fabrication of super-aligned nanofiber (SANF) scaffolds or sheets referred to as a Rotary Jet-Spinning Device or RJS device described in U.S. Patent Publication No. 2012/0135448 and PCT Publication No. WO 2012/068402, the entire contents of each of which are incorporated herein by reference. (B) Photographic image of an exemplary method for collecting super aligned nanofibers constructs from the reservoir. (C) Photographic image of scaffold constructs fabricated by rotary jet-spinning. (D) Representative scanning electron micrographs of cardiomyocytes, cortical neurons and valve interstitial cells cultured on super aligned polycaprolactone (PCL) and PCL/Collagen-75/25 biohybrid nanofiber scaffolds.

FIGS. 3A-3D depict an exemplary method for the assembly and operation of the a device of the invention. (A) Biohybrid nanofibers are fabricated by rotary jet-spinning and assembled into a nanofiber scaffold. (B) Scaffolds are seeded with skeletal muscle cells for culture, alignment and maturation. (C) A hydrogel precursor containing quiescent satellite muscle cells is applied on top of the engineered skeletal muscle and interpenetrates with the nanofiber scaffold upon gelification, thereby providing a continuous matrix and bringing into biochemical contact the skeletal and satellite muscle cells. (D) Laser cut horizontal polymeric fiber-engineered tissue assembled from the fiber-gel composite whose radius of curvature is measured optically for high throughput contractility experiments.

### DETAILED DESCRIPTION OF THE INVENTION

Described herein are devices, constructs and methods for measurements of physiologically relevant properties of in vitro tissue constructs. The devices and methods of the present invention can be used to measure muscle activities or functions, e.g., biomechanical forces that result from stimuli that include, but are not limited to, muscle cell contraction, osmotic swelling, structural remodeling and tissue level pre-stress. The devices and methods of the present invention may also be used for the evaluation of muscle activities or functions, e.g., electrophysiological responses, in a non-invasive manner, for example, in a manner that avoids cell damage. The devices and methods of the present invention are also useful for investigating muscle cell developmental biology and disease pathology, as well as in drug discovery and toxicity testing.

The benefits of the devices, constructs, and methods of the invention include, for example, creation of a microenvironment that more closely resembles an in vivo microenvironment, increasing the number of assays that may be performed simultaneously while decreasing the amount of test compound required, and the ability to create a wide range of test compound concentrations for simultaneous assaying of test compounds.

The benefit of the polymeric fiber scaffolds is that they may be finely tuned to mimic the mechanical properties of both healthy and diseased tissue, e.g., cardiac tissue.

The devices of the invention also permit longer-term culture of muscle tissue. For example, the tissues remain viable and spontaneously contract for about 5, 6, 7, 8, 9, 10, 11, or 12 days, while the devices of the invention comprising hydrogels remain viable and spontaneous contract for at least about 14, 15, 16, 17, 18, 19, 20, 21, 22, 23, 24, 25, 26, 27, 28, 29, 30, 31, 32, 33, 34, 35, or 36 days.

Furthermore, polymeric fibers and/or hydrogels do not absorb drugs applied to the muscle tissue and, therefore, do not interfere with assessment of the effect of the drug on a muscle tissue function.

### I. Devices and Constructs of the Invention and Methods of Production of the Same

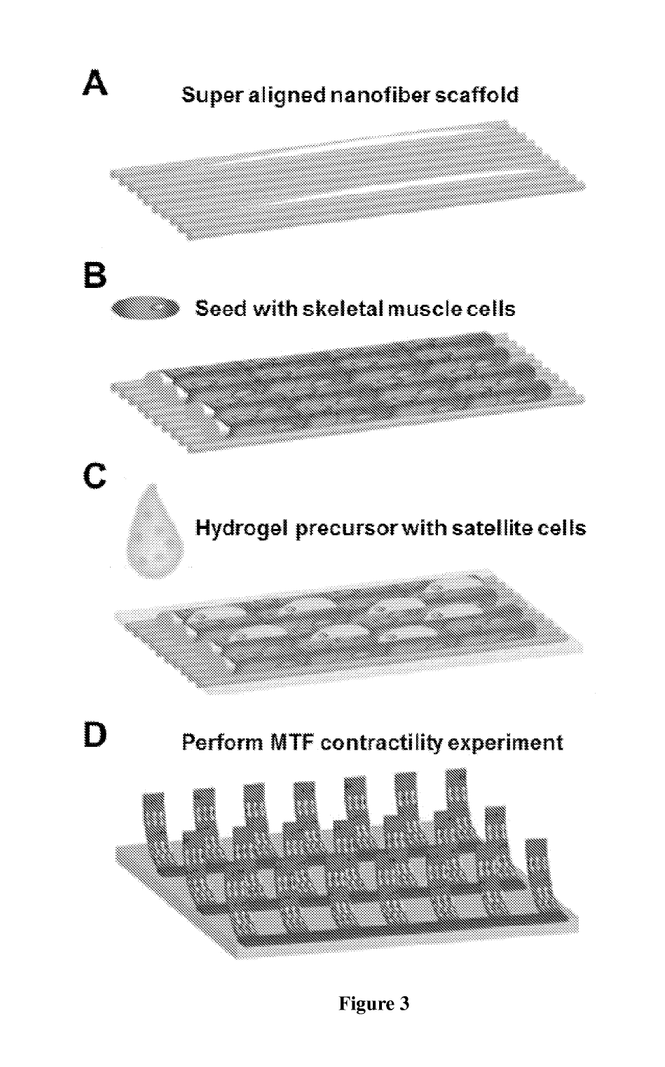

In one aspect, the present invention provides devices, e.g., devices for measuring a contractile function. The devices include a solid support structure, and a strip of co-cultured muscle tissue adhered to the solid support structure. The co-cultured muscle tissue comprises a layer of isolated cells seeded on a sheet of aligned polymeric fibers comprising a biogenic polymer, and a hydrogel layer comprising cells coated on the polymeric fiber layer and the strip of co-cultured muscle tissue can perform a contractile function. An exemplary device of the invention is depicted in FIG. 3D.

In some embodiments of the invention, the device comprises a plurality of strips of the co-cultured muscle tissue.

The present invention also provides constructs for producing a polymeric fibescaffolded engineered tissue. The constructs include a support structure, a sheet of aligned polymeric fibers on the support structure, wherein the aligned polymeric fibers comprise a biogenic polymer, cells seeded on the aligned polymeric fiber layer, and a hydrogel comprising cells coated on the aligned polymeric fiber layer seeded with cells.

The solid support structure may be formed of a rigid or semi-rigid material, such as a plastic, metal, ceramic, or a combination thereof. In one embodiment, the solid support structure is transparent so as to facilitate observation. In another embodiment, the solid support structure is opaque (e.g., light-absorbing). In one embodiment, a portion of the solid support structure is transparent (i.e., a portion underneath a portion of the co-cultured muscle tissue) and the remaining portion is opaque. In yet another embodiment, the solid support structure is translucent.

The solid support structure is ideally biologically inert, has low friction with the tissues and does not interact (e.g., chemically) with the tissues. Examples of materials that can be used to form the solid support structure include polystyrene, polycarbonate, polytetrafluoroethylene (PTFE), polyethylene terephthalate, quartz, silicon, and glass.

Suitable support structures for embodiments of the present invention include, for example, Petri dishes, cover-slips (circular or rectangular), strips of glass, glass slides, multi-well plates, microfluidic chambers, and microfluidic devices.

In another embodiment, the invention provides a microfluidics device comprising a solid support structure which comprises a plurality of co-cultured muscle tissue strips. In one embodiment, the plurality of microfluidic chambers is operably connected to two or more inlet microchannels each comprising a valve, such as described in, for example, WO 2007/044888, to regulate flow, and two or more outlet microchannels.

In one embodiment, the two or more inlet microchannels comprise one or more mixing chambers (a section of the inlet microchannel that generates turbidity). Such devices may have 2-1002 microchambers comprising a co-cultured muscle tissue of the invention, and 2, 3, 4, 5, 6, 7, 8, 9, or 10 inlet microchannels, each with a valve. Such devices may have from 1-1000 mixing chambers. Such devices are useful for generating concentration gradients of a test compound to perform a dose response assay with the test compound. The number of concentrations of the test compound that may be produced in such a device is dependent on the number of mixing chambers.

In another embodiment, the plurality of microfluidic chambers comprising a co-cultured muscle tissue of the invention is operably connected to one or more inlet ports and does not comprise a mixing chamber. Such devices may comprise 1-1000 inlet ports and 1-1000 microchambers comprising a co-cultured muscle tissue of the invention. Such devices are also useful for performing a dose response assay with a test compound, however the various drug concentrations must be pre-mixed and introduced intoan inlet port separately.

In one embodiment, the microfluidics devices of the invention further optionally comprise one or more (e.g., 1, 2, 3, 4, 5, 6, 7, 8, 9, or 10) collection ports.

Fluid may be moved through the microfluidics devices by any suitable means, such as electrochemical or pressure-driven means.

A microfluidic chamber and a microfluidic channel may be fabricated into one or more materials including but not limited to, Polydimethylsiloxane (PDMS), polyurethanes, other elastomers, thermoplastics (e.g. polymethyl methacrylate (PMMA), polyethylene, polyethylene terephthalate, polystyrene), epoxies and other thermosets, silicon, silicon dioxide, and indium tin oxide (ITO).

Any suitable method may be used to fabricate a microfluidic channel and/or chamber, such as, for example, micromachining, injection molding, laser etching, laser cutting, and soft lithography. In one embodiment, an electrode is fabricated into a chamber using a non-reactive metal, such as, platinum, gold, and indium tin oxide.

Sheets or scaffolds of biogenic polymeric fibers for use in the devices, constructs and methods of the invention are super-aligned, or those that comprise a plurality of fibers arrayed in substantially all the same direction (e.g., uniaxially aligned). In certain embodiments of the invention, the sheets or scaffolds of biogenic polymeric fibers may be mixtures of two or more polymers and/or two or more copolymers. In one embodiment the polymers may be a mixture of one or more polymers and or more copolymers. In another embodiment, the polymers for use in the devices and methods of the invention may be a mixture of one or more synthetic polymers and one or more naturally occurring polymers.

Any suitable method may be used to prepare the scaffolds. An exemplary method, referred to as Rotary-Jet Spinning (RJS) is described in Section II, below, and in U.S. Patent Publication No. 2012/0135448 and PCT Publication No. WO 2012/068402, the entire contents of each of which are incorporated herein by reference.

The terms “fiber” and “polymeric fiber” are used herein interchangeably, and both terms refer to fibers having micron, submicron, and nanometer dimensions.

Any suitable biogenic and/or non-biogenic polymer may be used to fabricate polymeric fiber sheets or scaffolds. Exemplary polymers for use in the devices, constructs, and methods of the invention may be biocompatible or non-biocompatible, synthetic or natural and those such as those that are synthetically designed to have shear induced unfolding.

Suitable synthetic polymers include, for example, poly(urethanes), poly(siloxanes) or silicones, poly(ethylene), poly(vinyl pyrrolidone), poly(2-hydroxy ethyl methacrylate), poly(N-vinyl pyrrolidone), poly(methyl methacrylate), poly(vinyl alcohol), poly(acrylic acid), polyacrylamide, poly(ethylene-co-vinyl acetate), poly(ethylene glycol), poly(methacrylic acid), polylactides (PLA), polyglycolides (PGA), poly(lactide-co-glycolides) (PLGA), polyanhydrides, polyphosphazenes, polygermanes, polyorthoesters, polyesters, polyamides, polyolefins, polycarbonates, polyaramides, polyimides, copolymers and derivatives thereof, and combinations thereof.

Suitable biogenic polymers, include, for example, proteins, polysaccharides, lipids, nucleic acids or combinations thereof.

Exemplary biogenic polymers, e.g., fibrous proteins, for use in the devices, constructs and methods of the invention include, but are not limited to, extracellular matrix proteins, silk (e.g., fibroin, sericin, etc.), keratins (e.g., alpha-keratin which is the main protein component of hair, horns and nails, beta-keratin which is the main protein component of scales and claws, etc.), elastins (e.g., tropoelastin, etc.), fibrillin (e.g., fibrillin-1 which is the main component of microfibrils, fibrillin-2 which is a component in elastogenesis, fibrillin-3 which is found in the brain, fibrillin-4 which is a component in elastogenesis, etc.), fibrinogen/fibrins/thrombin (e.g., fibrinogen which is converted to fibrin by thrombin during wound healing), fibronectin, laminin, collagens (e.g., collagen I which is found in skin, tendons and bones, collagen II which is found in cartilage, collagen III which is found in connective tissue, collagen IV which is found in extracellular matrix (ECM) protein, collagen V which is found in hair, etc.), vimentin, neurofilaments (e.g., light chain neurofilaments NF-L, medium chain neurofilaments NF-M, heavy chain neurofilaments NF-H, etc.), amyloids (e.g., alpha-amyloid, beta-amyloid, etc.), actin, myosins (e.g., myosin I-XVII, etc.), titin which is the largest known protein (also known as connectin), etc.

Exemplary biogenic polymers, e.g., fibrous polysaccharides, for use in the devices, constrcuts, and methods of the invention include, but are not limited to, chitin which is a major component of arthropod exoskeletons, hyaluronic acid which is found in extracellular space and cartilage (e.g., D-glucuronic acid which is a component of hyaluronic acid, D-N-acetylglucosamine which is a component of hyaluronic acid, etc.), etc.

Exemplary glycosaminoglycans (GAGs)—carbohydrate polymers found in the body—for use in the devices, constructs, and methods of the invention include, but are not limited to, heparan sulfate founding extracelluar matrix, chondroitin sulfate which contributes to tendon and ligament strength, keratin sulfate which is found in extracellular matrix, etc.

In certain embodiments of the invention, a biologically active agent, e.g., a polypeptide, protein, nucleic acid molecule, nucleotide, lipid, biocide, antimicrobial, or pharmaceutically active agent, may be mixed with the polymer during the fabrication process of the polymeric fibers. In other embodiments, a biologically inert agent, e.g., fluorescent beads, e.g., fluorospheres, may be mixed with the polymer during the fabrication process.

In yet another embodiment, polymers for use in the polymeric fibers of the invention are naturally occurring polymers, e.g., biogenic polymers. Non-limiting examples of such naturally occurring polymers include, for example, polypeptides, proteins, e.g., capable of fibrillogenesis, polysaccharides, e.g., alginate, lipids, nucleic acid molecules, and combinations thereof.

Any suitable hydrogel may be used in the devices, constructs, and methods of the invention and include, for example, biocompatible hydrogels comprising a substance, such as, but not limited to align, alignate, gelatin, fibrin, collagen, arginine, fibronectin, glucose, and a glycoprotein, or a combination thereof.

The cells on the aligned polymeric fiber sheet and in the hydrogel may be the same type of cells or different types of cells.

Examples of cell types that may be used include contractile cells, such as, but not limited to, vascular smooth muscle cells, vascular endothelial cells, myocytes (e.g., cardiac myocytes), skeletal muscle, myofibroblasts, airway smooth muscle cells and cells that will differentiate into contractile cells (e.g., stem cells, e.g., embryonic stem cells or adult stem cells, progenitor cells or satellite cells).

The term “progenitor cell” is used herein to refer to cells that have a cellular phenotype that is more primitive (e.g., is at an earlier step along a developmental pathway or progression than is a fully differentiated cell) relative to a cell which it can give rise to by differentiation. Often, progenitor cells also have significant or very high proliferative potential. Progenitor cells can give rise to multiple distinct differentiated cell types or to a single differentiated cell type, depending on the developmental pathway and on the environment in which the cells develop and differentiate.

The term “progenitor cell” is used herein synonymously with “stem cell.”

The term “stem cell” as used herein, refers to an undifferentiated cell which is capable of proliferation and giving rise to more progenitor cells having the ability to generate a large number of mother cells that can in turn give rise to differentiated, or differentiable daughter cells. The daughter cells themselves can be induced to proliferate and produce progeny that subsequently differentiate into one or more mature cell types, while also retaining one or more cells with parental developmental potential. The term “stem cell” refers to a subset of progenitors that have the capacity or potential, under particular circumstances, to differentiate to a more specialized or differentiated phenotype, and which retains the capacity, under certain circumstances, to proliferate without substantially differentiating. In one embodiment, the term stem cell refers generally to a naturally occurring mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell may derive from a multipotent cell which itself is derived from a multipotent cell, and so on. While each of these multipotent cells may be considered stem cells, the range of cell types each can give rise to may vary considerably. Some differentiated cells also have the capacity to give rise to cells of greater developmental potential. Such capacity may be natural or may be induced artificially upon treatment with various factors. In many biological instances, stem cells are also “multipotent” because they can produce progeny of more than one distinct cell type, but this is not required for “stem-ness.” Self-renewal is the other classical part of the stem cell definition. In theory, self-renewal can occur by either of two major mechanisms. Stem cells may divide asymmetrically, with one daughter retaining the stem state and the other daughter expressing some distinct other specific function and phenotype. Alternatively, some of the stem cells in a population can divide symmetrically into two stems, thus maintaining some stem cells in the population as a whole, while other cells in the population give rise to differentiated progeny only. Formally, it is possible that cells that begin as stem cells might proceed toward a differentiated phenotype, but then “reverse” and re-express the stem cell phenotype, a term often referred to as “dedifferentiation” or “reprogramming” or “retrodifferentiation”.

The term “embryonic stem cell” is used to refer to the pluripotent stem cells of the inner cell mass of the embryonic blastocyst (see U.S. Pat. Nos. 5,843,780, 6,200,806, the contents of which are incorporated herein by reference). Such cells can similarly be obtained from the inner cell mass of blastocysts derived from somatic cell nuclear transfer (see, for example, U.S. Pat. Nos. 5,945,577, 5,994,619, 6,235,970, which are incorporated herein by reference). The distinguishing characteristics of an embryonic stem cell define an embryonic stem cell phenotype. Accordingly, a cell has the phenotype of an embryonic stem cell if it possesses one or more of the unique characteristics of an embryonic stem cell such that that cell can be distinguished from other cells. Exemplary distinguishing embryonic stem cell characteristics include, without limitation, gene expression profile, proliferative capacity, differentiation capacity, karyotype, responsiveness to particular culture conditions, and the like.

The term “adult stem cell” or “ASC” is used to refer to any multipotent stem cell derived from non-embryonic tissue, including fetal, juvenile, and adult tissue. Stem cells have been isolated from a wide variety of adult tissues including blood, bone marrow, brain, olfactory epithelium, skin, pancreas, skeletal muscle, and cardiac muscle. Each of these stem cells can be characterized based on gene expression, factor responsiveness, and morphology in culture. Exemplary adult stem cells include neural stem cells, neural crest stem cells, mesenchymal stem cells, hematopoietic stem cells, and pancreatic stem cells.

In one embodiment, progenitor cells suitable for use in the claimed devices and methods are Committed Ventricular Progenitor (CVP) cells as described in PCT Application No. PCT/US09/060224, entitled “Tissue Engineered Mycocardium and Methods of Productions and Uses Thereof”, filed Sep. 28, 2009, the entire contents of which are incorporated herein by reference.

In one embodiment the cells are myocytes, e.g., cardiomyocytes. In another embodiment, the cells are smooth muscle cells or striated muscle cells. In another embodiment, the cells are muscle satellite cells. In one embodiment, the cells on the aligned polymeric fiber sheet are skeletal muscle cells and the cells in the hydrogel are muscle satellite cells.

The devices and constructs of the invention, and those for use in the methods of the invention are fabricated by providing a solid support structure and a sheet of aligned polymeric fibers on the solid support structure. The polymeric fiber layer is deposited on the solid support structure, i.e., is placed or applied onto the solid support structure. The polymeric fiber layer may be deposited on substantially the entire surface or only a portion of the surface of the solid support structure.

Cells are seeded on the aligned polymeric fiber layer and may or may not be cultured prior to applying a hydrogel comprising cells. In some embodiment, the cells seeded on the polymeric fiber layer are cultured for about 1 hour, 5 hours, 10 hours, 24 hours, or about 48 hours prior to applying the hydrogel comprising cells. In all cases, cells are cultured to form a tissue comprising, for example, anisotropic muscle cells and muscle satellite cells.

The hydrogel is applied as a hydrogel precursor, e.g., the hydrogel is poured onto the polymeric layer comprising cells, and subsequently interpenetrates the polymeric fiber layer. In some embodiments, fluorescent beads, e.g., fluorospheres, are mixed with the hydrogel prior to applying to the polymeric fiber layer.

The cells on are cultured in an incubator under physiologic conditions (e.g., at 37° C.) until the cells form a tissue.

Any appropriate cell culture method may be used to establish the tissue. The seeding density of the cells will vary depending on the cell size and cell type, but can easily be determined by methods known in the art. In one embodiment, cardiac myocytes are seeded at a density of between about 1×10⁵ to about 6×10⁵ cells/cm², or at a density of about 1×10⁴, about 2×10⁴, about 3×10⁴, about 4×10⁴, about 5×10⁴, about 6×10⁴, about 7×10⁴, about 8×10⁴, about 9×10⁴, about 1×10⁵, about 1.5×10⁵, about 2×10⁵, about 2.5×10⁵, about 3×10⁵, about 3.5×10⁵, about 4×10⁵, about 4.5×10⁵, about 5×10⁵, about 5.5×10⁵, about 6×10⁵, about 6.5×10⁵, about 7×10⁵, about 7.5×10⁵, about 8×10⁵, about 8.5×10⁵, about 9×10⁵, about 9.5×10⁵, about 1×10⁶, about 1.5×10⁶, about 2×10⁶, about 2.5×10⁶, about 3×10⁶, about 3.5×10⁶, about 4×10⁶, about 4.5×10⁶, about 5×10⁶, about 5.5×10⁶, about 6×10⁶, about 6.5×10⁶, about 7×10⁶, about 7.5×10⁶, about 8×10⁶, about 8.5×10⁶, about 9×10⁶, or about 9.5×10⁶. Values and ranges intermediate to the above-recited values and ranges are also contemplated by the present invention.

A portion of the formed tissue is removed, e.g., using a scalpel, razor blade, punch, die or laser, and strips, of the formed tissue including the polymeric layer adhered at one end, e.g., like a hinge, to the solid support structure are generated. The strips are free to deform or contract as a hinge. This allows the tissue to curve upward off the base layer, i.e., to curve upward from the viewing (horizontal plane), when stimulated to contract (see, e.g., FIG. 3D). Individual strips (e.g., 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100 or more strips) can be prepared on a single solid support structure, e.g., a glass cover slip (round or rectangular), a Petri dish, a glass slide, strips of glass, or a multi-well plate. The functional properties of these strips, e.g., the contractility of these strips, may be determined as described below.

A stimulus may be applied to the tissue to cause stress in the cell layer. The curvature of the tissue may recorded and cell stress is calculated. A fluid perfusion system can be used to wash out test compounds that are being screened in a high throughput assay or to refresh the culture medium.

The deformation (i.e., contractility) of the tissue may be recorded. In the embodiment, contractility may be observed (and optionally recorded) using a microscope, which looks at one strip at a time while it scans across multiple samples. In one embodiment of the invention, multiple strips are observed simultaneously. Optionally, a lens is integrated into the platform. Changes in the curvature of the films are observed and the optical image is converted to a numerical value that corresponds to the curvature of the tissue. In one embodiment, a movie of the tissue contractions is acquired (e.g., images are obtained in series). Images are processed and a mechanical analysis is optionally carried out to evaluate contractility. The output may be traction as a function of standard metrics such as peak systolic stress, peak upstroke power, upstroke time, and relaxation time.

Alternative ways of measuring contractility of the engineered co-cultured tissues include, e.g., (i) using a laser bounced off of the thin film to record movement, (ii) using an integrated piezoelectric film in the tissue and recording a change in voltage during bending, (iii) integrating magnetic particles in the polymeric fibers and measuring the change in magnetic field during bending, (iv) placing a lens in the bottom of each well and simultaneously projecting multiple wells onto a single detector (e.g., camera, CCD or CMOS) at one time, (v) using a single capture device to sequentially record each well (e.g., the capture device is placed on an automated motorized stage. Finally, the measured bending information (e.g., digital image or voltage) is converted into force, frequency and other contractility metrics.

In one embodiment, the methods for fabricating a polymeric fiber-scaffolded engineered tissue, further comprise attaching a multi-well plate skeleton to the solid support structure prior to cell culture.

In one embodiment, the devices of the invention further comprises a photodiode array.

In one embodiment, the solid support structure may further comprise an optical signal capture device and an image processing software to calculate change in an optical signal. The optical signal capture device may further include fiber optic cables in contact with the device and/or a computer processor in contact with the device.

In one embodiment, an electrode is in contact with the device.

In the embodiments of the invention where the solid support structure is a multi-well plate, each well may contain one strip of tissue, two, or multiple strips of tissue.

In certain embodiments of the invention, e.g., for evaluation of electrophysiological activities, cells are cultured in the presence of a fluorophor such as a voltage-sensitive dye or an ion-sensitive dye. For example, the voltage-sensitive dye is an electrochromic dye such as a a styryl dye or a merocyanine dye. Exemplary electrochromic dyes include RH-421 or di-4-ANEPPS. Ion-sensitive, e.g., calcium sensitive dyes, include aequorin, Fluo3, and Rhod2. For simultaneous measurements of action potentials and intracellular calcium, the following exemplary dye pairs are used: di-2-ANEPEQ and calcium green; di-4-ANEPPS and Indo-1; di-4-ANEPPS and Fluo-4; RH237 and Rhod2; and, RH-237 and Fluo-3/4.

In such embodiments, the device includes strip of tissue grown in multi-well, e.g., 2- 8-, 12-, 16-, 20-, 24-, 28-, 32- 36-, 40, 44, 48-, 96-, 192-, 384-well, plates prepared as described herein. An inverted microscope or contact-fluorescence imaging system with temperature-controlled, humidity-controlled motorized may be used to monitor muscle activity, e.g., electrophysiological changes, such as action potentials and/or intracellular calcium transients. An integrated fluid-handling system may also be used to apply/exchange fluorophores and test compounds, and a microfluidics chamber may be used for simulated drug delivery. The microfluidics chamber simulates microvasculature to mimic the manner in which a compound/drug contacts a target strip of tissue comprising, e.g., myocytes.

Appropriate light source and filter sets may be chosen for each desired fluorophore based on the wavelength of the excitation light and fluoresced light of the fluorophore. Integration of excitation wavelength-switching or an additional detector permits ratiometric calcium imaging. For this purpose, exemplary fluorophores include Fura-2 and Indo-1 or Fluo-3 and Fura Red. For example, excitation and emission filters at 515±5 and >695 nm, respectively, are used to measure action potentials with di-4-ANEPPS, and excitation and emission filters at 365±25 and 485±5 nm, respectively, are used to measure calcium transients with Indo-1. Automated software may be used and customized for data acquisition and data analysis.

Advantages of the optical mapping system include non-invasiveness (no damage is inflicted to the cell membrane), recorded signals are real-time action potentials and/or calcium transients in contrast to derivatives of action potentials like extracellular recordings or slowly changing intracellular ionic concentrations or membrane potential like the FLIPR system.

For high-throughput optical mapping, analysis may be carried out using two different imaging approaches. For Contact Fluorescence Mapping, a microscope is not required. Fiber optic cables contact the bottom of a culture plate or wells of a multi-well plate containing the tissue strips. The plate or wells of the plate are then mapped based on the detected fluorescence. To screen compounds, test compounds are added to each individual well of a multi-well plate, and each bundle of fiber optic cables collects data from each different well providing data pertaining to tissue response to the test compound.

In another embodiment, an inverted microscope may be used to map each well individually. Cells of a tissue strip are contacted with, e.g., a chromophore, a fluorophor, or a bioluminescent material, and the microscope objective is moved from well to well to measure muscle activities or functions, e.g., electrophysiological changes. For example, the response of the tissue strip to each test compound is monitored for alterations in cardiac excitation, e.g., to identify drugs that induce or do not cause cardiac arrhythmia. Each of the approaches provides significant advantages (e.g., speed, efficiency, no or minimal user contact with the tissue strip, reduced user skill required, ability to observe and measure cell-cell interactions, ability to map action potential propagation and conduction velocity, and ability to observe and measure fibrillation and arrhythmia)) compared to previous assays used to measure electrophysiological changes (e.g., patch clamp assay in which a single cell is patch clamped).

These systems are well suited to screen test compounds for, for example, cardiac safety. For example, FDA Guideline S7B addresses “Safety pharmacology studies for assessing the potential for delayed ventricular repolarization by human pharmaceuticals”. The devices and high-throughput in vitro assays described herein allow the identification of cardiac safety risks much earlier in the drug discovery process. The devices and methods of the invention are also useful for anti-arrhythmic and/or ion channel-targeted drug discovery.

### II. Aligned Polymeric Fiber Scaffolds

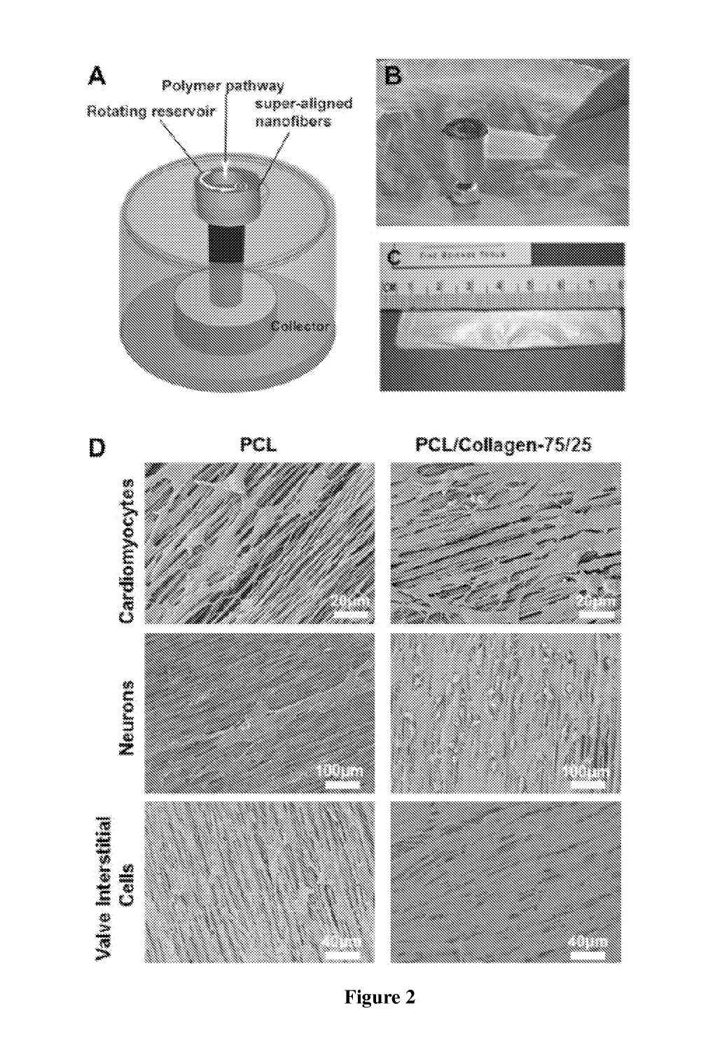

Scaffolds of aligned biogenic polymer fibers, e.g., polymeric fibers, suitable for use in the claimed devices, constructs, and methods may be prepared using a system and/or device employing rotational motion and without the use of an electric field e.g., a high voltage electrical field. Such devices are described in U.S. Patent Publication No. 2012/0135448 and in PCT Publication No. WO 2012/068402, the entire contents of each of which are incorporated herein by reference. Devices employing rotational motion for the preparation of polymeric fibers are referred to herein as “Rotary Jet Spinning Devices” or “RJS Devices.” An exemplary RJS device is depicted in FIG. 2A.

Exemplary devices for the preparation of polymeric fibers for use in the claimed devices, constructs, and methods may include one or more reservoirs for containing a material solution for forming the polymeric fibers having micron, submicron, and nanometer dimensions, and one or more collection devices for collecting the formed fibers employing rotational motion.

The reservoir and collection device may be constructed of any material, e.g., a material that can withstand heat and/or that is not sensitive to chemical organic solvents.

The reservoir and the collection device may be made of a plastic material, e.g., polypropylene, polyethylene, and polytetrafluoroethylene, or a metal, e.g., aluminum, steel, stainless steel, tungsten carbide, tungsten alloys, titanium and nickel.

Any suitable size or geometrically shaped reservoir or collector may be used. For example, the reservoir may be round, rectangular, or oval.

An RJS device may further comprise a component suitable for continuously feeding the polymer into the reservoir, such as a spout or syringe pump.

In certain embodiments, the collection device is maintained at about room temperature, e.g., about 20, 21, 22, 23, 24, 25, 26, 27, 28, 29, or about 30° C. and ambient humidity, e.g., about 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or about 90% humidity.

The devices may be maintained at and the methods may be formed at any suitable temperature and humidity depending on the desired surface topography of the polymeric fibers to be fabricated. For example, increasing humidity from about 30% to about 50% results in the fabrication of porous fibers, while decreasing humidity to about 25% results in the fabrication of smooth fibers. As smooth fibers have more tensile strength than porous fibers, in one embodiment, the devices of the invention are maintained and fibers are prepared in controlled humidity conditions, e.g., humidity varying by about less than about 10%.

The reservoir may also include a heating element for heating and/or melting the polymer.

In an exemplary RJS Device, an exemplary reservoir includes one or more orifices through which a material solution may be ejected from the reservoir during fiber formation. The devices include sufficient orifices for ejecting the polymer during operation, such as 1, 2, 3, 4, 5, 6, 7, 8, 9, 10, 11, 12, 13, 14, 15, or more orifices.

The orifices may be provided on any surface or wall of the reservoir, e.g., side walls, top walls, bottom walls, etc. In exemplary embodiments in which multiple orifices are provided, the orifices may be grouped together in close proximity to one another, e.g., on the same surface of the reservoir, or may be spaced apart from one another, e.g., on different surfaces of the reservoir.

The orifices may be of the same diameter or of different diameters, e.g., diameters of about 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1.0, 2, 3, 4, 5, 6, 7, 8, 9, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or about 1000 micrometers.

Diameters intermediate to the above-recited values are also intended to be part of this invention.

The length of the one or more orifices may be the same or different, e.g., diameters of about 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, or 0.1 m. Lengths intermediate to the above recited lengths are also contemplated to be part of the invention.

One or more jets of a material solution are ejected from one or more reservoirs containing the material solution, and one or more air foils are used to modify the air flow and/or air turbulence in the surrounding air through which the jets of the material solution descend which, in turn, affects the alignment of the fibers that are formed from the jets.

Rotational speeds of the reservoir may range from about 1,000 rpm-50,000 rpm, about 1,000 rpm to about 40,000 rpm, about 1,000 rpm to about 20,000 rpm, about 5,000 rpm-20,000 rpm, about 5,000 rpm to about 15,000 rpm, or about 50,000 rpm to about 400,000 rpm, e.g., about 1,000, 1,500, 2,000, 2,500, 3,000, 3,500, 4,000, 4,500, 5,000, 5,500, 6,000, 6,500, 7,000, 7,500, 8,000, 8,500, 9,000, 9,500,10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, 20,000, 20,500, 21,000, 21,500, 22,000, 22,500, 23,000, 23,500, or about 24,000, 50,000, 55,000, 60,000, 65,000, 70,000, 75,000, 80,000, 85,000, 90,000, 95,000, 100,000, 105,000, 110,000, 115,000, 120,000, 125,000, 130,000, 135,000, 140,000, 145,000, 150,000 rpm, about 200,000 rpm, 250,000 rpm, 300,000 rpm, 350,000 rpm, or 400,000 rpm. Ranges and values intermediate to the above recited ranges and values are also contemplated to be part of the invention.

In certain embodiments, rotating speeds of about 50,000 rpm-400,000 rpm are employed. In one embodiment, devices employing rotational motion may be rotated at a speed greater than about 50,000 rpm, greater than about 55,000 rpm, greater than about 60,000 rpm, greater than about 65,000 rpm, greater than about 70,000 rpm, greater than about 75,000 rpm, greater than about 80,000 rpm, greater than about 85,000 rpm, greater than about 90,000 rpm, greater than about 95,000 rpm, greater than about 100,000 rpm, greater than about 105,000 rpm, greater than about 110,000 rpm, greater than about 115,000 rpm, greater than about 120,000 rpm, greater than about 125,000 rpm, greater than about 130,000 rpm, greater than about 135,000 rpm, greater than about 140,000 rpm, greater than about 145,000 rpm, greater than about 150,000 rpm, greater than about 160,000 rpm, greater than about 165,000 rpm, greater than about 170,000 rpm, greater than about 175,000 rpm, greater than about 180,000 rpm, greater than about 185,000 rpm, greater than about 190,000 rpm, greater than about 195,000 rpm, greater than about 200,000 rpm, greater than about 250,000 rpm, greater than about 300,000 rpm, greater than about 350,000 rpm, or greater than about 400,000 rpm.

Rotation is for a time sufficient to form a desired polymeric fiber, such as, for example, about 1 minute to about 100 minutes, about 1 minute to about 60 minutes, about 10 minutes to about 60 minutes, about 30 minutes to about 60 minutes, about 1 minute to about 30 minutes, about 20 minutes to about 50 minutes, about 5 minutes to about 20 minutes, about 5 minutes to about 30 minutes, or about 15 minutes to about 30 minutes, about 5-100 minutes, about 10-100 minutes, about 20-100 minutes, about 30-100 minutes, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 minutes, or more. Times and ranges intermediate to the above-recited values are also intended to be part of this invention.

Suitable biogenic polymer fiber sheets or scaffolds are formed using the devices by providing a volume of a polymer solution and imparting a shear force to a surface of the polymer solution such that the polymer in the solution is unfolded, thereby forming a fiber.

In one embodiment, the polymer solution is a biogenic polymer solution. In one embodiment, the shear force is sufficient to expose molecule-molecule, e.g., protein-protein, binding sites in the polymer, thereby inducing fibrillogenesis.

### III. Methods and Uses of the Devices of the Invention

The devices of the invention are useful for, among other things, measuring muscle activities or functions, investigating muscle developmental biology and disease pathology, as well as in drug discovery and toxicity testing.

Accordingly, the present invention also provides methods for identifying a compound that modulates a contractile function. The methods include providing a polymeric fiber-scaffolded engineered tissue; contacting the polymeric fiber-scaffolded engineered tissue with a test compound; and determining the effect of the test compound on a contractile function in the presence and absence of the test compound, wherein a modulation of the contractile function in the presence of the test compound as compared to the contractile function in the absence of the test compound indicates that the test compound modulates a contractile function, thereby identifying a compound that modulates a contractile function.

In another aspect, the present invention also provides methods for identifying a compound useful for treating or preventing a muscle disease. The methods include providing a polymeric fiber-scaffolded engineered tissue; contacting the polymeric fiber-scaffolded engineered tissue with a test compound; and determining the effect of the test compound on a contractile function in the presence and absence of the test compound, wherein a modulation of the contractile function in the presence of the test compound as compared to the contractile function in the absence of the test compound indicates that the test compound modulates a contractile function, thereby identifying a compound useful for treating or preventing a muscle disease.

The methods of the invention generally comprise determining the effect of a test compound on a polymeric fiber-scaffolded engineered tissue as a whole, however, the methods of the invention may comprise further evaluating the effect of a test compound on an individual cell type(s) of the polymeric fiber-scaffolded engineered tissue.

As used herein, the various forms of the term “modulate” are intended to include stimulation (e.g., increasing or upregulating a particular response or activity) and inhibition (e.g., decreasing or downregulating a particular response or activity).

As used herein, the term “contacting” (e.g., contacting a polymeric fiber-scaffolded engineered tissue with a test compound) is intended to include any form of interaction (e.g., direct or indirect interaction) of a test compound and a polymeric fiber-scaffolded engineered tissue or a plurality of polymeric fiber-scaffolded engineered tissue s. The term contacting includes incubating a compound and a polymeric fiber-scaffolded engineered tissue or plurality of polymeric fiber-scaffolded engineered tissues together (e.g., adding the test compound to a polymeric fiber-scaffolded engineered tissue or plurality of polymeric fiber-scaffolded engineered tissues in culture).

Test compounds, may be any agents including chemical agents (such as toxins), small molecules, pharmaceuticals, peptides, proteins (such as antibodies, cytokines, enzymes, and the like), nanoparticles, and nucleic acids, including gene medicines and introduced genes, which may encode therapeutic agents, such as proteins, antisense agents (i.e., nucleic acids comprising a sequence complementary to a target RNA expressed in a target cell type, such as RNAi or siRNA), ribozymes, and the like.

The test compound may be added to a polymeric fiber-scaffolded engineered tissue by any suitable means. For example, the test compound may be added drop-wise onto the surface of a device of the invention and allowed to diffuse into or otherwise enter the device, or it can be added to the nutrient medium and allowed to diffuse through the medium. In the embodiment where the device of the invention comprises a multi-well plate, each of the culture wells may be contacted with a different test compound or the same test compound. In one embodiment, the screening platform includes a microfluidics handling system to deliver a test compound and simulate exposure of the microvasculature to drug delivery. In one embodiment, a solution comprising the test compound may also comprise fluorescent particles, and a muscle cell function may be monitored using Particle Image Velocimetry (PIV).

Numerous physiologically relevant parameters, e.g., muscle activities, e.g., biomechanical and electrophysiological activities, can be evaluated using the methods and devices of the invention. For example, in one embodiment, the devices of the present invention can be used in contractility assays for contractile cells, such as muscular cells or tissues, such as chemically and/or electrically stimulated contraction of vascular, airway or gut smooth muscle, cardiac muscle, vascular endothelial tissue, or skeletal muscle. In addition, the differential contractility of different muscle cell types to the same stimulus (e.g., pharmacological and/or electrical) can be studied.

In another embodiment, the devices of the present invention can be used for measurements of solid stress due to osmotic swelling of cells. For example, as the cells swell the polymeric fiber-scaffolded engineered tissue will bend and as a result, volume changes, force and points of rupture due to cell swelling can be measured.

In another embodiment, the devices of the present invention can be used for pre-stress or residual stress measurements in cells. For example, vascular smooth muscle cell remodeling due to long term contraction in the presence of endothelin-1 can be studied.

Further still, the devices of the present invention can be used to study the loss of rigidity in tissue structure after traumatic injury, e.g., traumatic brain injury. Traumatic stress can be applied to vascular smooth muscle thin films as a model of vasospasm. These devices can be used to determine what forces are necessary to cause vascular smooth muscle to enter a hyper-contracted state. These devices can also be used to test drugs suitable for minimizing vasospasm response or improving post-injury response and returning vascular smooth muscle contractility to normal levels more rapidly.

In other embodiments, the devices of the present invention can be used to study biomechanical responses to paracrine released factors (e.g., vascular smooth muscle dilation due to release of nitric oxide from vascular endothelial cells, or cardiac myocyte dilation due to release of nitric oxide).

In other embodiments, the devices of the invention can be used to evaluate the effects of a test compound on an electrophysiological parameter, e.g., an electrophysiological profile comprising a voltage parameter selected from the group consisting of action potential, action potential morphology, action potential duration (APD), conduction velocity (CV), refractory period, wavelength, restitution, bradycardia, tachycardia, reentrant arrhythmia, and/or a calcium flux parameter, e.g., intracellular calcium transient, transient amplitude, rise time (contraction), decay time (relaxation), total area under the transient (force), restitution, focal and spontaneous calcium release, and wave propagation velocity. For example, a decrease in a voltage or calcium flux parameter of a polymeric fiber-scaffolded engineered tissue comprising cardiomyocytes upon contacting the polymeric fiber-scaffolded engineered tissue with a test compound, would be an indication that the test compound is cardiotoxic.

In yet another embodiment, the devices of the present invention can be used in pharmacological assays for measuring the effect of a test compound on the stress state of a tissue. For example, the assays may involve determining the effect of a drug on tissue stress and structural remodeling of the polymeric fiber-scaffolded engineered tissue. In addition, the assays may involve determining the effect of a drug on cytoskeletal structure (e.g., sarcomere alignment) and, thus, the contractility of the polymeric fiber-scaffolded engineered tissue.

In still other embodiments, the devices of the present invention can be used to measure the influence of biomaterials on a biomechanical response. For example, differential contraction of vascular smooth muscle remodeling due to variation in material properties (e.g., stiffness, surface topography, surface chemistry or geometric patterning) of polymeric thin films can be studied.

In further embodiments, the devices of the present invention can be used to study functional differentiation of stem cells (e.g., pluripotent stem cells, multipotent stem cells, induced pluripotent stem cells, and progenitor cells of embryonic, fetal, neonatal, juvenile and adult origin) into contractile phenotypes. For example, undifferentiated cells, e.g., stem cells, are coated on the thin films and differentiation into a contractile phenotype is observed by thin film bending. Differentiation into an anisotropic tissue may also be observed by quantifying the degree of alignment of sarcomeres and/or quantifying the orientational order parameter (OOP). Differentiation can be observed as a function of: co-culture (e.g., co-culture with differentiated cells), paracrine signaling, pharmacology, electrical stimulation, magnetic stimulation, thermal fluctuation, transfection with specific genes, chemical and/or biomechanical perturbation (e.g., cyclic and/or static strains).

In another embodiment, the devices of the invention may be used to determine the toxicity of a test compound by evaluating, e.g., the effect of the compound on an electrophysiological response of a polymeric fiber-scaffolded engineered tissue. For example, opening of calcium channels results in influx of calcium ions into the cell, which plays an important role in excitation-contraction coupling in cardiac and skeletal muscle fibers. The reversal potential for calcium is positive, so calcium current is almost always inward, resulting in an action potential plateau in many excitable cells. These channels are the target of therapeutic intervention, e.g., calcium channel blocker sub-type of anti-hypertensive drugs. Candidate drugs may be tested in the electrophysiological characterization assays described herein to identify those compounds that may potentially cause adverse clinical effects, e.g., unacceptable changes in cardiac excitation, that may lead to arrhythmia.

For example, unacceptable changes in cardiac excitation that may lead to arrhythmia include, e.g., blockage of ion channel requisite for normal action potential conduction, e.g., a drug that blocks Na⁺ channel would block the action potential and no upstroke would be visible; a drug that blocks Ca²⁺ channels would prolong repolarization and increase the refractory period; blockage of K⁺ channels would block rapid repolarization, and, thus, would be dominated by slower Ca²⁺ channel mediated repolarization.

In addition, metabolic changes may be assessed to determine whether a test compound is toxic by determining, e.g., whether contacting with a test compound results in a decrease in metabolic activity and/or cell death. For example, detection of metabolic changes may be measured using a variety of detectable label systems such as fluormetric/chrmogenic detection or detection of bioluminescence using, e.g., AlamarBlue fluorescent/chromogenic determination of REDOX activity (Invitrogen), REDOX indicator changes from oxidized (non-fluorescent, blue) state to reduced state(fluorescent, red) in metabolically active cells; Vybrant MTT chromogenic determination of metabolic activity (Invitrogen), water soluble MTT reduced to insoluble formazan in metabolically active cells; and Cyquant NF fluorescent measurement of cellular DNA content (Invitrogen), fluorescent DNA dye enters cell with assistance from permeation agent and binds nuclear chromatin. For bioluminescent assays, the following exemplary reagents is used: Cell-Titer Glo luciferase-based ATP measurement (Promega), a thermally stable firefly luciferase glows in the presence of soluble ATP released from metabolically active cells.

The devices of the invention are also useful for evaluating the effects of particular delivery vehicles for therapeutic agents e.g., to compare the effects of the same agent administered via different delivery systems, or simply to assess whether a delivery vehicle itself (e.g., a viral vector or a liposome) is capable of affecting the biological activity of the polymeric fiber-scaffolded engineered tissue. These delivery vehicles may be of any form, from conventional pharmaceutical formulations, to gene delivery vehicles. For example, the devices of the invention may be used to compare the therapeutic effect of the same agent administered by two or more different delivery systems (e.g., a depot formulation and a controlled release formulation). The devices and methods of the invention may also be used to investigate whether a particular vehicle may have effects of itself on the tissue. As the use of gene-based therapeutics increases, the safety issues associated with the various possible delivery systems become increasingly important. Thus, the devices of the present invention may be used to investigate the properties of delivery systems for nucleic acid therapeutics, such as naked DNA or RNA, viral vectors (e.g., retroviral or adenoviral vectors), liposomes and the like. Thus, the test compound may be a delivery vehicle of any appropriate type with or without any associated therapeutic agent.

Furthermore, the devices of the present invention are a suitable in vitro model for evaluation of test compounds for therapeutic activity with respect to, e.g., a muscular and/or neuromuscular disease or disorder. For example, the devices of the present invention (e.g., comprising muscle cells) may be contacted with a candidate compound by, e.g., diffusion of the test compound added drop-wise on the surface of a polymeric fiber-scaffolded engineered tissue, diffusion of a test compound through the culture medium, or immersion in a bath of media containing the test compound, and the effect of the test compound on muscle activity (e.g., a biomechanical and/or electrophysiological activity) may measured as described herein, as compared to an appropriate control, e.g., an untreated polymeric fiber-scaffolded engineered tissue. Alternatively, a device of the invention may be bathed in a medium containing a candidate compound, and then the cells are washed, prior to measuring a muscle activity (e.g., a biomechanical and/or electrophysiological activity) as described herein. Any alteration to an activity determined using the device in the presence of the test agent (as compared to the same activity using the device in the absence of the test compound) is an indication that the test compound may be useful for treating or preventing a muscle disease, e.g., a neuromuscular disease.

For use in the methods of the invention, the cells seeded onto the polymeric fiber-scaffolded engineered tissue may be normal muscle cells (cardiac, smooth, or skeletal muscle cells), abnormal muscle cells (e.g., those derived from a diseased tissue, or those that are physically or genetically altered to achieve a abnormal or pathological phenotype or function), normal or diseased muscle cells derived from embryonic stem cells or induced pluripotent stem cells, or normal cells that are seeded/printed onto the film in an abnormal or aberrant configuration. In some cases, both muscle cells and neuronal cells are present on the film.

Evaluation of muscle activity includes determining the degree of contraction, i.e., the degree of curvature or bend of the muscular film, and the rate or frequency of contraction/rate of relaxation compared to a normal control or control film in the absence of the test compound. An increase in the degree of contraction or rate of contraction indicates that the compound is useful in treatment or amelioration of pathologies associated with myopathies such as muscle weakness or muscular wasting. Such a profile also indicates that the test compound is useful as a vasocontractor. A decrease in the degree of contraction or rate of contraction is an indication that the compound is useful as a vasodilator and as a therapeutic agent for muscle or neuromuscular disorders characterized by excessive contraction or muscle thickening that impairs contractile function.

Compounds evaluated in this manner are useful in treatment or amelioration of the symptoms of muscular and neuromuscular pathologies such as those described below. Muscular Dystrophies include Duchenne Muscular Dystrophy (DMD) (also known as Pseudohypertrophic), Becker Muscular Dystrophy (BMD), Emery-Dreifuss Muscular Dystrophy (EDMD), Limb-Girdle Muscular Dystrophy (LGMD), Facioscapulohumeral Muscular Dystrophy (FSH or FSHD) (Also known as Landouzy-Dejerine), Myotonic Dystrophy (MMD) (Also known as Steinert's Disease), Oculopharyngeal Muscular Dystrophy (OPMD), Distal Muscular Dystrophy (DD), and Congenital Muscular Dystrophy (CMD). Motor Neuron Diseases include Amyotrophic Lateral Sclerosis (ALS) (Also known as Lou Gehrig's Disease), Infantile Progressive Spinal Muscular Atrophy (SMA, SMA1 or WH) (also known as SMA Type 1, Werdnig-Hoffman), Intermediate Spinal Muscular Atrophy (SMA or SMA2) (also known as SMA Type 2), Juvenile Spinal Muscular Atrophy (SMA, SMA3 or KW) (also known as SMA Type 3, Kugelberg-Welander), Spinal Bulbar Muscular Atrophy (SBMA) (also known as Kennedy's Disease and X-Linked SBMA), Adult Spinal Muscular Atrophy (SMA). Inflammatory Myopathies include Dermatomyositis (PM/DM), Polymyositis (PM/DM), Inclusion Body Myositis (IBM). Neuromuscular junction pathologies include Myasthenia Gravis (MG), Lambert-Eaton Syndrome (LES), and Congenital Myasthenic Syndrome (CMS). Myopathies due to endocrine abnormalities include Hyperthyroid Myopathy (HYPTM), and Hypothyroid Myopathy (HYPOTM). Diseases of peripheral nerves include Charcot-Marie-Tooth Disease (CMT) (Also known as Hereditary Motor and Sensory Neuropathy (HMSN) or Peroneal Muscular Atrophy (PMA)), Dejerine-Sottas Disease (DS) (Also known as CMT Type 3 or Progressive Hypertrophic Interstitial Neuropathy), and Friedreich's Ataxia (FA). Other Myopathies include Myotonia Congenita (MC) (Two forms: Thomsen's and Becker's Disease), Paramyotonia Congenita (PC), Central Core Disease (CCD), Nemaline Myopathy (NM), Myotubular Myopathy (MTM or MM), Periodic Paralysis (PP) (Two forms: Hypokalemic—HYPOP—and Hyperkalemic—HYPP) as well as myopathies associated with HIV/AIDS.

The methods and devices of the present invention are also useful for identifying therapeutic agents suitable for treating or ameliorating the symptoms of metabolic muscle disorders such as Phosphorylase Deficiency (MPD or PYGM) (Also known as McArdle's Disease), Acid Maltase Deficiency (AMD) (Also known as Pompe's Disease), Phosphofructokinase Deficiency (PFKM) (Also known as Tarui's Disease), Debrancher Enzyme Deficiency (DBD) (Also known as Cods or Forbes' Disease), Mitochondrial Myopathy (MITO), Carnitine Deficiency (CD), Carnitine Palmityl Transferase Deficiency (CPT), Phosphoglycerate Kinase Deficiency (PGK), Phosphoglycerate Mutase Deficiency (PGAM or PGAMM), Lactate Dehydrogenase Deficiency (LDHA), and Myoadenylate Deaminase Deficiency (MAD).

In addition to the disorders listed above, the screening methods described herein are useful for identifying agents suitable for reducing vasospasms, heart arrhythmias, and cardiomyopathies.

Vasodilators identified as described above are used to reduce hypertension and compromised muscular function associated with atherosclerotic plaques. Smooth muscle cells associated with atherosclerotic plaques are characterized by an altered cell shape and aberrant contractile function. Such cells are used to populate a thin film, exposed to candidate compounds as described above, and muscular function evaluated as described above. Those agents that improve cell shape and function are useful for treating or reducing the symptoms of such disorders.

Smooth muscle cells and/or striated muscle cells line a number of lumen structures in the body, such as uterine tissues, airways, gastrointestinal tissues (e.g., esophagus, intestines) and urinary tissues, e.g., bladder. The function of smooth muscle cells on thin films in the presence and absence of a candidate compound may be evaluated as described above to identify agents that increase or decrease the degree or rate of muscle contraction to treat or reduce the symptoms associated with a pathological degree or rate of contraction. For example, such agents are used to treat gastrointestinal motility disorders, e.g., irritable bowel syndrome, esophageal spasms, achalasia, Hirschsprung's disease, or chronic intestinal pseudo-obstruction.

The present invention is next described by means of the following examples. However, the use of these and other examples anywhere in the specification is illustrative only, and in no way limits the scope and meaning of the invention or of any exemplified form. The invention is not limited to any particular preferred embodiments described herein. Many modifications and variations of the invention may be apparent to those skilled in the art and can be made without departing from its spirit and scope. The contents of all references, patents and published patent applications cited throughout this application, including the figures, are incorporated herein by reference.

### EXAMPLES

### Example 1

### nFAST Skeletal Muscle on a Chip

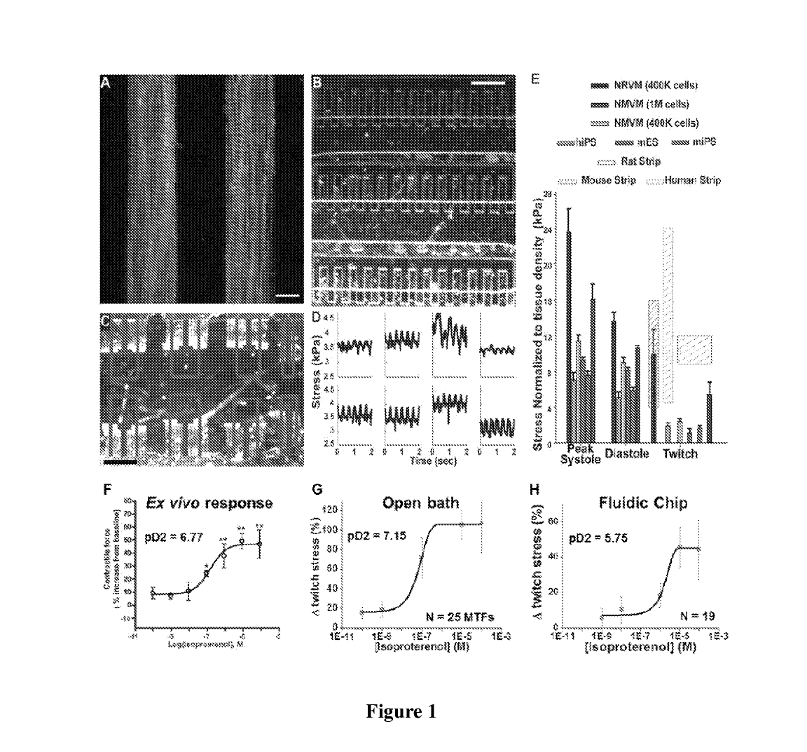

Previously, a biohybrid system for engineering muscle and measuring muscular contractions that exploited the surface chemistry of polydimethylsiloxane (PDMS) to precisely engineer laminar striated and smooth muscle was have developed (see, e.g., U.S. Patent Publication Nos. 2009/0317852 and 2012/0142556, the entire contents of each of which are incorporated herein by reference (see, e.g., FIG. 1). This system was amenable to parallel arrays of muscular constructs in microfluidic chambers, automated measurements of contractility data and drug wash-in and wash-out experiments. With ventricular cardiac muscle, it was demonstrated that this assay could replicate contractility data and dose response measured in isolated adult rat ventricular strips. This system is fast, easy to use, and amenable to traditional 2D culture techniques commonly used in the pharmaceutical and biotechnology industries.

With the next generation of this technology, a system is fabricated which is 1) amenable to both 2D- and 3D-engineered tissue samples, 2) replaces the synthetic polymer thin film with extracellular matrix, and is 3) amenable to heterogeneous cell demographics. The functional ease of the cantilever bending optical readout described for the 2D-system is retained in the 3D-system.

Previously, a unique method for making nanofibers that replaces electrospinning, Rotary Jet Spinning (RJS), was developed (Badrossamay, et al., 2010) and was shown to induce the unfolding of globular extracellular matrix proteins such as fibronectin through centrifugal and shear forces to induce fibrillogenesis and the mass production of nanofibers (FIG. 2). As indicated in FIG. 2B, super-aligned nanofibers can be prepared.

When used as a scaffold for engineered tissues, biodegradeable polymers or hybrid materials of biodegradeable synthetic (FIG. 2D) and natural biological polymers (data not shown) may be used to produce 2D or 3D sengineered tissues. These materials support the growth of muscle, neuronal and valve interstitial cells, inducing cell alignment and, in the case of neurons, directed extension of axons. This fiber manufacturing technique is thus amenable to 2D systems for higher throughput screening, but is scaleable to 3D tissue constructs.

Using the nanofibers, arrayed as a scaffold for tissue (nFAST) a 2D anisotropic muscle scaffold is prepared (FIG. 3). The nanofiber array is built with RJS, then seeded with skeletal muscle cells. By having the muscle cells on the apical side of the 2D nFAST, electrically-stimulated contraction will induce a vertical displacement of the nFAST. The benefit of this design is that because of the scaffolds' modular design, additional cell types may be introduced in the form of a cell-doped hydrogel. In the first version of this, satellite cells are used and their integration into the muscular tissue is determined. Arrays of the muscular nFAST can be used during drug experiments and, time in culture may be extended from days to weeks because of the natural scaffolding material. Automated data acquisition, as previously developed for the MTF technology, is applicable here with minimal modification because of the differences in the mechanical properties of the scaffolding materials.

### EQUIVALENTS

In describing embodiments of the invention, specific terminology is used for the sake of clarity. For purposes of description, each specific term is intended to at least include all technical and functional equivalents that operate in a similar manner to accomplish a similar purpose. Additionally, in some instances where a particular embodiment of the invention includes a plurality of system elements or method steps, those elements or steps may be replaced with a single element or step; likewise, a single element or step may be replaced with a plurality of elements or steps that serve the same purpose. Further, where parameters for various properties are specified herein for embodiments of the invention, those parameters can be adjusted up or down by 1/20th, 1/10th, ⅕th, ⅓rd, ½, etc., or by rounded-off approximations thereof, unless otherwise specified. Moreover, while this invention has been shown and described with references to particular embodiments thereof, those skilled in the art will understand that various substitutions and alterations in form and details may be made therein without departing from the scope of the invention; further still, other aspects, functions and advantages are also within the scope of the invention. The contents of all references, including patents and patent applications, cited throughout this application are hereby incorporated by reference in their entirety. The appropriate components and methods of those references may be selected for the invention and embodiments thereof. Still further, the components and methods identified in the Background section are integral to this disclosure and can be used in conjunction with or substituted for components and methods described elsewhere in the disclosure within the scope of the invention.

1. A device for measuring a contractile function, the device comprising:
a solid support structure; and
a strip of co-cultured muscle tissue adhered to the solid support structure, wherein the co-cultured muscle tissue comprises a layer of isolated cells seeded on a sheet of aligned polymeric fibers comprising a biogenic polymer, and a hydrogel layer comprising cells coated on the polymeric fiber layer, wherein the strip of co-cultured muscle tissue can perform a contractile function.

2. The device of claim 1, comprising a plurality of strips of the co-cultured muscle tissue.

3. The device of claim 1, wherein the cells on the aligned polymeric fiber sheet and in the hydrogel are of the same type, or are different types of cells.

4. (canceled)

5. The device of claim 1, wherein the cells are selected from the group consisting of myocytes, cardiomyocytes, smooth muscle cells, striated muscle cells, and muscle satellite cells.

6.-8. (canceled)

9. The device of claim 1, wherein the cells on the aligned polymeric fiber sheet are skeletal muscle cells and the cells in the hydrogel are muscle satellite cells.

10.-14. (canceled)

15. The device of claim 1, wherein the aligned polymeric fiber sheet is prepared by rotary jet-spinning.

16. The device of claim 1, wherein the biogenic polymer is a protein, a polysaccharide, a lipid, a nucleic acid, or a combination thereof.

17.-19. (canceled)

20. The device of claim 1, wherein the polymeric fiber is a biohybrid fiber.

21. (canceled)

22. A construct for producing a polymeric fiber-scaffolded engineered tissue comprising:
a support structure;
a sheet of aligned polymeric fibers on the support structure, wherein the aligned polymeric fibers comprise a biogenic polymer;
cells seeded on the aligned polymeric fiber layer; and
a hydrogel comprising cells coated on the aligned polymeric fiber layer seeded with cells.

23. The construct of claim 22, wherein the cells on the aligned polymeric fiber sheet and in the hydrogel are the same type of cells, or different types of cells.

24. (canceled)

25. The construct of claim 22, wherein the cells are selected from the group consisting of myocytes, cardiomyocytes, smooth muscle cells, striated muscle cells, and muscle satellite cells.

26.-33. (canceled)

34. The construct of claim 22, wherein the aligned polymeric fiber sheet is prepared by rotary jet-spinning.

35. The construct of claim 22, wherein the biogenic polymer is a protein, a polysaccharide, a lipid, a nucleic acid, or a combination thereof.

36.-38. (canceled)

39. The construct of claim 22, wherein the polymeric fiber is a biohybrid fiber.

40. (canceled)

41. A method for fabricating a polymeric fiber-scaffolded engineered tissue comprising:
providing a solid support structure;
providing a sheet of aligned polymeric fibers on the solid support structure, wherein the aligned polymeric fibers comprise an extracellular matrix protein;
seeding cells on the aligned polymeric fiber layer;
applying a hydrogel comprising cells on the cells seeded on the sheet of aligned polymeric fibers;
culturing the cells to form a tissue; and
removing a portion of said formed tissue thereby generating strips of said formed tissue adhered at one end to said solid support structure.

42. The method of claim 41, wherein the cells on the aligned polymeric fiber sheet and in the hydrogel are the same type of cells or different types of cells.

43. (canceled)

44. The method of claim 41, wherein the cells are selected from the group consisting of myocytes, cardiomyocytes, smooth muscle cells, striated muscle cells, and muscle satellite cells.

45.-52. (canceled)

53. The method of claim 41, wherein the aligned polymeric fiber sheet is prepared by rotary jet-spinning.

54. The method of claim 41, wherein the biogenic polymer is a protein, a polysaccharide, a lipid, a nucleic acid, or a combination thereof.

55.-57. (canceled)

58. The construct of claim 41, wherein the polymeric fiber is a biohybrid fiber.

59. (canceled)

60. A polymeric fiber-scaffolded engineered tissue prepared according to the method of claim 41.

61. A method for identifying a compound that modulates a contractile function, the method comprising
providing a polymeric fiber-scaffolded engineered tissue;
contacting the polymeric fiber-scaffolded engineered tissue with a test compound; and
determining the effect of the test compound on a contractile function in the presence and absence of the test compound, wherein a modulation of the contractile function in the presence of said test compound as compared to the contractile function in the absence of said test compound indicates that said test compound modulates a contractile function, thereby identifying a compound that modulates a contractile function.

62. A method for identifying a compound useful for treating or preventing a muscle disease, the method comprising providing a polymeric fiber-scaffolded engineered tissue;
contacting the polymeric fiber-scaffolded engineered tissue with a test compound; and
determining the effect of the test compound on a contractile function in the presence and absence of the test compound, wherein a modulation of the contractile function in the presence of said test compound as compared to the contractile function in the absence of said test compound indicates that said test compound modulates a contractile function, thereby identifying a compound useful for treating or preventing a muscle disease.

63. The method of claim 61, wherein the contractile function is a biomechanical activity or an electrophysiological activity.

64. (canceled)

65. The method of claim 62, wherein the contractile function is a biomechanical activity or an electrophysiological activity.

66.-68. (canceled)
